(19) United States
(12) Patent Application Publication
(10) Pub. No.: US 2008/0275501 A1
(43) Pub. Date: Nov. 6, 2008

(54) MEDICAL DEVICE LOADING AND DELIVERY SYSTEMS AND METHODS

(76) Inventors: Albert K. CHIN, Palo Alto, CA (US); Arthur M. Lin, Fremont, CA (US); Kenny Dang, San Jose, CA (US); Alfredo R. Cantu, Pleasanton, CA (US); Paul A. DeLipski, San Mateo, CA (US); Ali Eslambolchi, San Jose, CA (US); Kumar Jambunathan, Sunnyvale, CA (US); Kristopher Yee, San Jose, CA (US); Juan I. Perez, San Jose, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/113,518

(22) Filed: May 1, 2008

Related U.S. Application Data
(60) Provisional application No. 60/916,019, filed on May 4, 2007.

(51) Int. Cl. A61B 17/03

(52) U.S. Cl. 606/213

(57) ABSTRACT

Described herein are systems and methods for inserting a hemostatic seal into a delivery device. One such system can include a loading tool and a delivery device configured to detachably mate with one another. The loading tool can house a hemostatic seal in its unfolded configuration and can fold the seal for delivery into the delivery device. The delivery device can then be advanced into the loading tool to insert the seal into the delivery device.

This application claims priority to Provisional Application Ser. No. 60/916,019 entitled “Medical Device Loading And Delivery Systems And Methods” filed May 4, 2007, which is incorporated herein by reference.

### BACKGROUND OF THE INVENTION

Contemporary coronary artery bypass grafting surgery is performed on a beating heart to reduce complications commonly associated with the prior surgical practice of transitioning a patient onto and off of a heart-lung machine. Performing an aortotomy and a proximal anastomisis on an aorta that is perfused with pressurized blood can be facilitated with temporary sealing methods to curtail blood flow through the aortic hole. Side-bite and surface-oriented clamping mechanisms have been used to diminish blood loss during such procedures, but such temporary occlusions can damage the endothelium and dislodge emboli that may migrate through the circulatory system. Alternative schemes for performing an aortotomy and limiting loss of blood include introducing a plug or seal at the site of the aortotomy, but such schemes commonly inhibit convenient and rapid completion of the graft anastomosis.

In response, new methods and instrumentation have been developed. In accordance with these advancements, an aorto-coronary bypass graft is performed by puncturing the aortic wall and inserting a hemostatic sheath that selectively delivers and positions a seal within the aortic hole. The seal is retented against the aortic wall under tension established by an external structure. The suture anastomosis is performed with the hemostatic seal in place and with a central stem of the seal residing near the location of the last placed stitch. The seal is then removed as a tear-away strip that is pulled, initially by the seal stem, through a removal instrument. Additional discussion of these procedures and instrumentation can be found in U.S. Pat. No. 6,814,743, and application Ser. Nos. 10/123,470 and 10/952,392. These disclosures are also incorporated herein by reference.

The seal, in its fully deployed shape, is too large to easily fit through the aortic hole or in the delivery sheath through which it is delivered. Therefore, use of the seal requires it be manually rolled and partially inserted into the delivery tube immediately prior to insertion into the aortotomy. The seal, which is usually formed of a polyurethane coated surgical suture, wound in a spiraling fashion and heat molded to retain its shape, is easily cracked or permanently deformed during manipulation. Further, the seal cannot be packaged and provided pre-loaded in the delivery tube, as it takes set into the rolled up configuration and does not deploy properly to cover the aortotomy if it is distorted.

Accordingly, current surgical procedures could benefit from improved techniques and devices for folding the hemostatic seal shortly before its use and/or for inserting the folded seal into a delivery sheath.

### SUMMARY OF THE INVENTION

Described herein are methods, systems, and devices for loading and delivering a hemostatic seal. One exemplary system includes an anatomic seal comprising a flexible body and a loading tool. The loading tool can house the seal prior to configuring the seal for delivery into a delivery device. For example, the loading tool can include a seal housing area sized and shaped for housing a substantially unfolded seal. When a user desires to implant the seal, the loading tool can fold the seal and facilitate delivery into the delivery device.

In one aspect, the loading tool comprises an elongate body having an proximal opening and a distal seal housing area. The elongate body can further include a channel for receiving at least a portion of the delivery device. The seal housing area can include a folding mechanism comprising at least one articulating member having a seal contact surface.

In another aspect, a delivery device comprises an elongate body having a proximal and distal end. At least a portion of an outer surface of the delivery device can be sized and shaped for receipt within the loading tools. The delivery device can further comprise an open interior for receiving a folded seal. A plunger, positioned at least partially within the open interior, can deploy the seal. For example, the delivery device can include a plunger control mechanism for moving the plunger relative to the open interior. In another aspect, the delivery device can include a locking mechanism for preventing or allowing articulation of the plunger relative to the open interior.

In one aspect, the loading tool and delivery device are configured to detachably mate with one another. For example, at least a portion of the loading tool channel can have a size and shape corresponding to the delivery device. In addition, or alternatively, the loading tool and delivery device can mechanically and/or frictionally engage one another.

In order to facilitate loading of the folded seal into the delivery device, the loading tool can have a variety of alignment features to align the delivery device with the folded seal. In one aspect, the loading tool channel includes at least one alignment feature that allows relative longitudinal movement of the delivery device, but restricts relative movement in another direction (e.g., orthogonal). For example, the at least one alignment feature can allow the delivery device to move into and out of the loading tool channel by does not allow pivotal, side-to-side, and/or up-down movement of the delivery device with respect to the loading tool. Thus, in one embodiment, the alignment features prevent at least one degree of freedom, at least two degrees of freedom, or more than two degrees of freedom of the delivery device with respect to the loading tool.

In another aspect, the loading tool can include at least one stop to limit advancement of the delivery device into the loading tool. For example, the loading tool can include a channel having at least one cross-sectional distance that is smaller than a first width of the delivery device and larger than a second width of the delivery device. A user can advance the delivery device through the channel until the larger first width prevents further insertion. In another aspect, the systems described herein can include a soft stop that inhibits, but does not prevent relative movement between the delivery device and loading tool. The soft stops can reduce the chance of unwanted or accidental movement of the delivery device with respect to the loading tool.

In one aspect, the loading tool channel is adapted to allow passage of a portion of the delivery device, but not the seal when the seal is in an unfolded configuration. For example, the channel can include a cross-sectional width and/or shape that prevents passage of an unfolded seal and allows passage of a folded seal.

In another embodiment described herein, a lockable delivery device is provided. The delivery device can include an elongate body extending between a proximal handle and a distal opening, the elongate body including an inner lumen for receiving the hemostatic seal in a folded configuration. The delivery device can further comprise a plunger extending through at least a portion of the lumen and movably mated with the elongate body. In one aspect, a locking mechanism defined by at least a portion of the elongate body and/or plunger can prevent movement of the plunger with respect to the lumen when locked and configured to allow movement of the plunger with respect the lumen when unlocked.

### BRIEF DESCRIPTION OF THE DRAWINGS

The accompanying drawings, which are incorporated in and constitute a part of this specification, illustrate exemplary embodiments of the invention and, together with the description, serve to explain the principles of the invention.

FIG. 1 is a top view of one exemplary embodiment of a seal loading system described herein;

FIG. 2A is a front view of one exemplary embodiment of a hemostatic seal and loading tool system described herein;

FIG. 2B is a front view of one exemplary embodiment of a folded hemostatic seal described herein;

FIG. 3 is a partially disassembled view of one exemplary embodiment of a loading system described herein;

FIG. 4A is a plan view of one exemplary embodiment of the loading tool described herein;

FIG. 4B is a plan view of another exemplary embodiment of the device of FIG. 4A;

FIG. 5A is a plan view of another exemplary embodiment of a the loading tool described herein;

FIG. 5B is a partial plan view of another exemplary embodiment of the device of FIG. 5A;

FIG. 6A is a front view of an exemplary folding mechanism described herein;

FIG. 6B is a perspective view of the folding mechanism of FIG. 6A;

FIG. 7A is a cut-away view of an exemplary embodiment of the loading system described herein;

FIG. 7B is a cut-away view another exemplary embodiment of the system of FIG. 7A;

FIG. 8A is a top view of an exemplary embodiment of the delivery device described herein;

FIG. 8B is a side view of the delivery device of FIG. 8A;

FIG. 8C is a perspective view of the delivery device of FIG. 8A;

FIG. 9 is an exploded view of an exemplary embodiment of the delivery device described herein; and

FIG. 10 is a partial view of the one exemplary embodiment of the delivery device described herein.

### DESCRIPTION OF EXEMPLARY EMBODIMENTS

Disclosed herein are various methods and devices for implanting a medical device, and particularly, for configuring hemostatic seal for delivery, and delivering hemostatic seals. In one aspect, a seal loading device is described, and in another aspect, a seal delivery device is described. The seal loading device can fold the sealing element into a reduced size for insertion into a delivery area of the seal delivery device.

In another embodiment, various systems are described. In one aspect, the loading tool houses the seal and the seal is delivered to a clinician inside the loading tool. For example, the loading tool can include a seal housing area where the seal resides prior to configuring the seal for delivery. The housing area can be sized and shaped such that the seal is unfolded while positioned within the housing area. Shortly before use, the loading tool can fold the seal into a configuration for receipt in the delivery device.

In another aspect, a system comprises a loading tool detachably mated to a delivery device. The loading tool and delivery device can be delivered to a user as a single unit. In yet another embodiment, a system can include a seal, a loading tool, and a delivery device. The devices can be packaged and delivered to a user a single unit.

As mentioned above, in one embodiment, the seal is a hemostatic seal for use with a variety of procedures. Hemostatic sealing elements are generally known in the art and find use during cardiac procedures, such as coronary by-pass procedures, for sealing an aperture created in a beating heart. Such devices are generally folded in the operating room, deployed through a tissue aperture, and expanded within the heart to create a seal. Rather than pre-package the sealing elements in a folded configuration, a surgeon folds the hemostatic seal in the operating room, shortly before inserting the sealing element into a seal insertion instrument. In particular, with conventional seals, a surgeon will hand roll the sealing element and manually insert it into the insertion instrument. Unfortunately, this can be a burdensome procedure, sometimes resulting in several broken seals before proper folding and loading is achieved. The systems disclosed herein utilizes a seal loading system that facilitates the task of folding the sealing element and inserting it into the insertion instrument, thereby reducing the risk of sealing element damage and/or for reducing the amount of time necessary to prepare the seal.

Reference will now be made in detail to the exemplary embodiments of the invention, examples of which are illustrated in the accompanying drawings. Wherever possible, the same reference numbers will be used throughout the drawings to refer to the same or like parts.

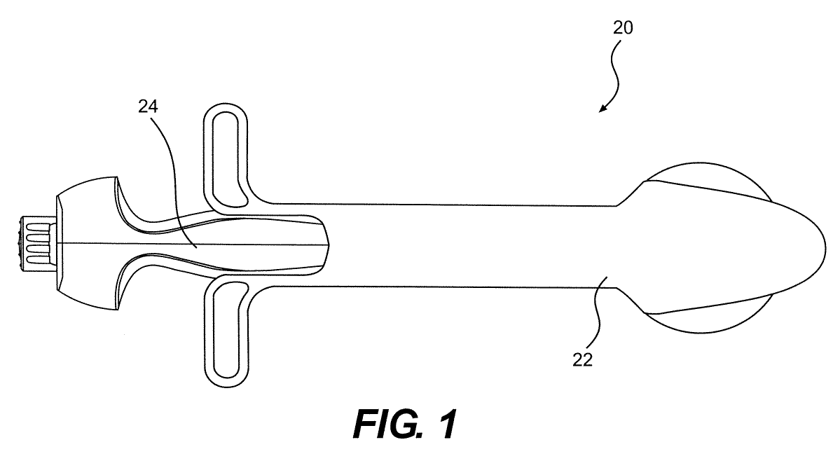

Referring now to FIG. 1, there is shown a seal insertion instrument system 20 comprising a loading tool 22 and delivery device 24. In one aspect, the loading tool and delivery device can detachably mate with one another. A seal positioned within loading tool 22 can be moved from a storage configuration into a delivery configuration while within the loading tool. The seal can then be inserted into the delivery device. In one aspect, the delivery device can be moved relative to the loading tool to insert the seal into a portion of delivery device. The delivery device can then be detached from the loading tool and used to implant the seal into a patient.

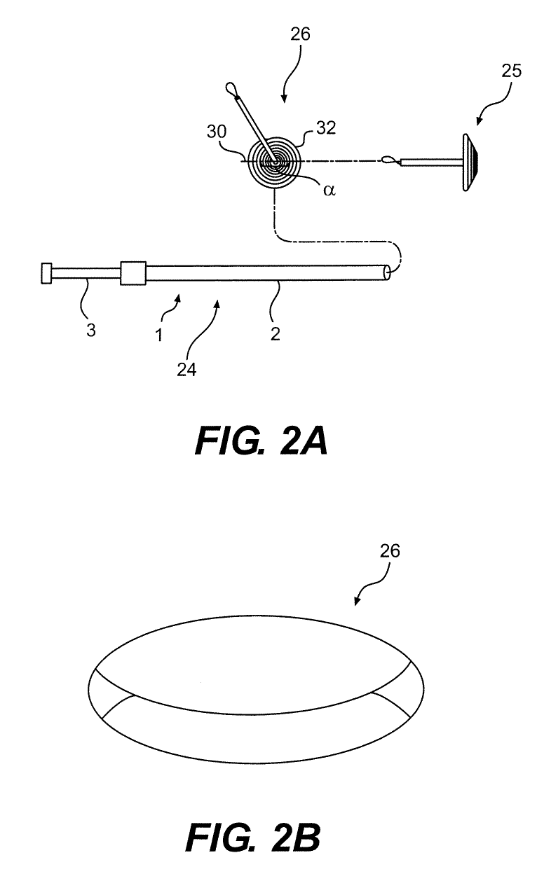

As illustrated in FIGS. 2A and 2B, sealing element 26, in its deployed state, is of a convex-concave or mushroom shape with an outer diameter larger than the aortic aperture to be sealed. Medical seals of various shapes, however, can be used with the seal loading apparatus described herein.

In order to insert sealing element 26 into an aortic aperture, the sealing element can be folded. As used herein, the term “fold” can refer to changing the size of at least one dimension of the seal. Thus, folding can refer to rolling, bending, and/or overlapping edges of the seal. In one exemplary aspect, the seal is folded by overlapping any two of the seal's non-adjacent edges. For example, in one aspect, as illustrated in FIG. 2A, edges 30, 32 are opposing edges, spaced by an angle α of 180 degrees. Alternatively, the edges can be spaced by at least 90 degrees, and in another aspect, spaced in the range of about 150 and 180 degrees from one another. This method of folding renders the previously mushroom-shaped sealing element in the shape of an oblate spheroid or ellipsoid with somewhat blunted or flattened ends. In latitudinal cross-section, the folded seal can be substantially circular. Longitudinally, the cross-section appears generally oval or ellipsoidal with blunted or flattened ends. This configuration is depicted in FIG. 2B. The terms “oblate spheroid” and “oblate ellipsoid” are not used here in their precise geometric sense. Rather, a section of these shapes represents a portion of the folded sealing element having a generally taco or football-like shape.

Once folded, seal 26 can be inserted, lengthwise, into an insertion instrument and, subsequently, through an aortic aperture, then released. The fully deployed seal, having thus resumed its mushroom shape, can then be anchored against the aperture through which it was inserted, sealing the aortic hole and preventing blood loss.

Further details regarding an exemplary sealing element 26 and additional embodiments of the aortic sealing element can be found in U.S. Pat. No. 6,814,743, and application Ser. Nos. 10/123,470 and 10/952,392. As mentioned above, however, the invention herein disclosed is not limited to use with any one of these seals and could be used in conjunction with seals of various shapes and sizes.

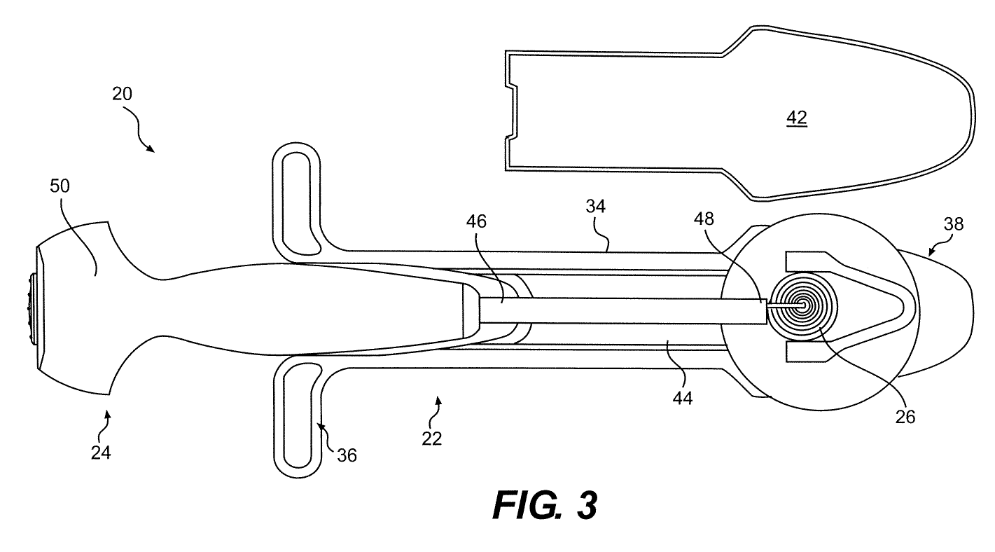

FIG. 3 illustrates an exemplary embodiment of system 20 including loading tool 22 having an elongate body 34 extending between a proximal end 36 and a distal end 38. In one aspect, body 34 is a multi-part structure including a main body portion and a cover 42. FIG. 3 illustrates body 34 with cover 42 removed to expose a portion of the inner surface 44 of body 34. Alternatively, body 34 could be formed of a singe piece structure or of multiple parts that are fixedly or detachably mated to one another. Regardless, seal 26 and a portion of delivery device 24 are positioned within body 34.

Delivery device 24 includes an elongate body 46 having a distal end 48 for mating with seal 26 and a proximal end 50 having controls and/or a handle. In one aspect, the delivery device body 46 includes an inner lumen sized and shaped for receiving a folded seal. The outer surface of delivery device 24 can be sized and shaped for mating with loading tool 22.

The loading tool and delivery device can be formed from the variety of biocompatible and/or medical grade materials including metals, polymers, and/or elastomers. In one aspect, at least a portion of the delivery device and/or loading tool is formed of a transparent or translucent material that allows a user to view the seal within the loading tool and/or delivery device. In use, the transparent material can define a seal visualization window that assists a user by allowing confirmation of proper folding and insertion of the seal into the delivery device. The full length of loading tool 22 need not be formed of transparent or translucent material, for example, only the distal portion of the loading tool containing seal 26 can include a window. In an alternative aspect, an opening in the body of the loading tool and/or delivery device can provide the window.

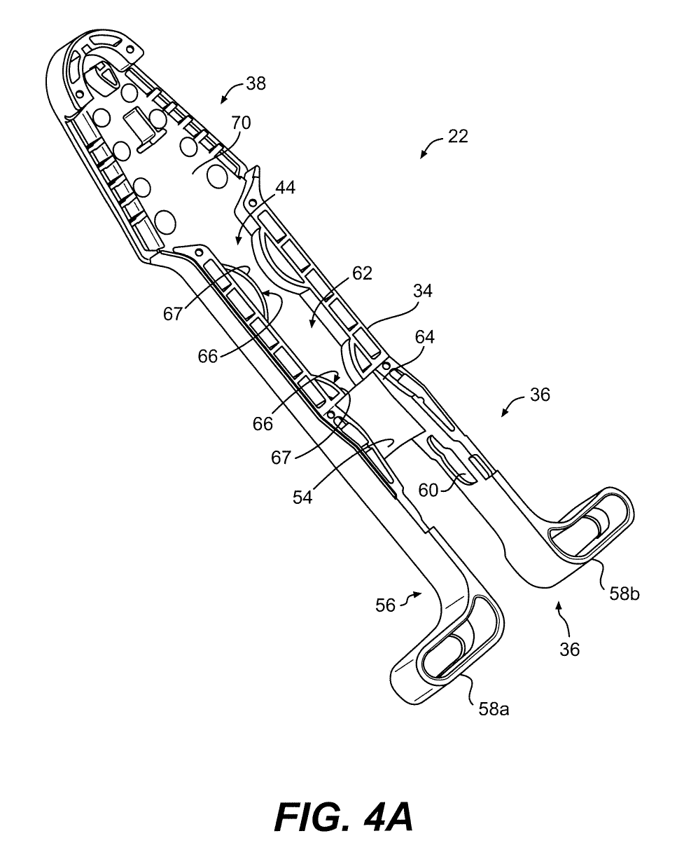

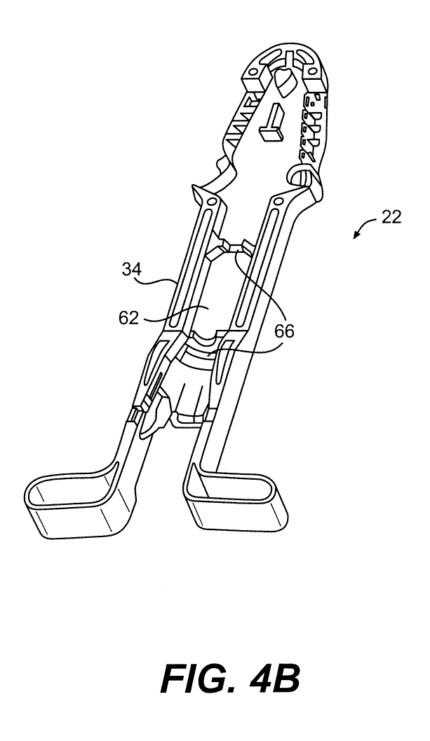

FIGS. 4A and 4B illustrate a cutaway or plan view of loading tool 22 showing inner surface 44 of body 34 with the upper portion of loading tool 22 removed. The loading tool can extend between a proximal end 36 and a distal end 38, where the proximal end 36 of the loading tool defines an opening 54 for receiving at least a portion of the delivery device. Proximate to opening 54, loading tool 22 can include proximal handle 56. In one aspect, handle 56 includes two “L” shaped members 58a, 58b that extend proximally from the loading tool. The inner surfaces of handle members 58a, 58b can help to align the delivery device by guiding the delivery device into opening 54. For example, the inner surfaces of handle members 58a, 58b can be spaced to match an outer diameter of at least a portion of the delivery device.

In one aspect, handle 56 is configured to facilitate relative movement between the loading tool and delivery device by allowing the user to apply generally opposing forces on the loading tool and deliver device. The user can grip handle 56 and a portion of the delivery device and move the delivery device distally into the loading tool and/or move the delivery device proximally to withdraw the delivery device from the loading tool. In one exemplary embodiment, handle 56 allow a syringe-like articulation of system 20. A user can grasp each handle members 58a, 58b with a finger and use a thumb (or palm) to move the delivery device into the loading tool. This configuration can, for example, allow single-hand control of system 20. One skilled in the art will appreciate that a variety of alternative handle configurations can be used with system 20. For example, while handle 56 is illustrated at the proximal-most end of loading tool 22, a user handle can be formed at a variety of locations along body 34 and can include a single handle member or more than two handles members.

The inner and/or outer surface of the loading tool can detachably mate with the outer surface of the delivery device. For example, the inner surface of the loading tool can house a portion of the delivery device. The detachably mated delivery device and loading tool can be mechanically and/or frictionally engaged to inhibit relative movement prior to a user articulating the delivery device.

For example, loading tool body 34 can include a variety of hard or soft stops to control relative movement of the delivery device and/or provide user feedback. Soft stops generally refer to features that increase or reduce the force necessary to move the delivery device relative to the loading tool, but do not prevent relative movement between the loading tool and delivery device. The soft stops can inhibit relative movement of the delivery device prior to preparation of the seal for insertion into the delivery device. In order to advance the delivery device into the loading tool, a user has to overcome the resistance provided by the soft stop.

One or more soft stops can be positioned at a variety of locations where the loading tool and delivery device contact one another. For example, proximate to opening 54, the loading tool can include a raised surface feature 60 that mates with a recessed or raised surface on the delivery device. Surface feature 60 increases resistance to relative distal and/or proximal movement of delivery device 24 and defines a soft stop. In one exemplary aspect, surface feature 60 and a corresponding recess on delivery device 24 require bending, flexing, and/or compression of surface feature 60 and/or handle members 58a, 58b to move the delivery device distally into the loading tool. The resistance to bending, flexing, and/or compression, along with the size and shape of the surface feature 60 and its corresponding recess, can be chosen based on the desired amount of resistance created by the soft stop.

Once the delivery device moves past the soft stop, resistance to relative movement between the loading tool and delivery device can decrease. However, in another aspect, resistance can be continuous or increasing with further advancement of the delivery device. In addition, the soft stop can signal a user, via tactile feedback, of the relative location of the delivery device with respect to the loading tool.

In addition to soft stops, the loading tool can include one or more hard stops that prevent relative movement between the loading tool and delivery device. The inner surface 44 of loading tool 22 can include a channel 62 through which the delivery device travels. A portion of channel 62 can have a diameter that prevents passage of a portion of the delivery device. For example, channel 62 can include a reduced diameter defined by a wall 64 that prevents a portion of the delivery device from further distal movement. As will be described in more detail below, system 20 can insert folded seal 26 only partially into the lumen of the delivery device. The hard stop can prevent over-insertion and/or damage to the seal.

Loading tool channel 62 can also serve to align the delivery device with the folded seal. One or more alignment features 66 can allow longitudinal movement (e.g., proximal/distal movement) between the loading tool and delivery device while inhibiting at least one degree of freedom of the delivery device with respect to the loading tool. In one aspect, alignment feature 66 has a size and shape that corresponds to the outer surface of a portion of the loading tool and limits relative transverse movement (i.e., side-to-side and/or up-down) and/or pivotal movement of the delivery device relative to the loading tool.

In one aspect, alignment feature 66 is an aperture defined by the inner surface of the loading tool. For example, as illustrated in FIG. 4B, loading tool 22 includes a generally curved or half-circle surface that corresponds to a generally circular outer surface of the delivery device. It should be appreciated that the alignment feature and delivery device can have alternative cross-sectional shapes, such as, for example, rectangular, oval, triangular, and/or irregular, which may, or may not correspond to one another. In another aspect, loading tool 22 includes more than one alignment aperture 66 that together limit relative lateral and pivotal movement of the delivery device with respect to the loading tool.

In another embodiment, the alignment feature or features can establish point contact with the delivery device. For example, as illustrated in FIG. 4A, the alignment features 66 can extend into channel 62 and include an inner-most surface 67 that contacts the delivery device. In one aspect, two, three, or more than three points around a circumference of the delivery device are in contact with alignment features.

The loading tool described herein can further comprise a seal housing area 70, in which the unfolded seal resides prior to folding and insertion into the delivery device. Seal housing area 70 can have a size and shape that allow the seal to remain substantially unfolded and can be defined by the body 34 of loading tool 22. For example, the width, length, and height of the seal housing area can be equal to or greater than a substantially unfolded seal. In one aspect, the seal is folded while in the seal housing area.

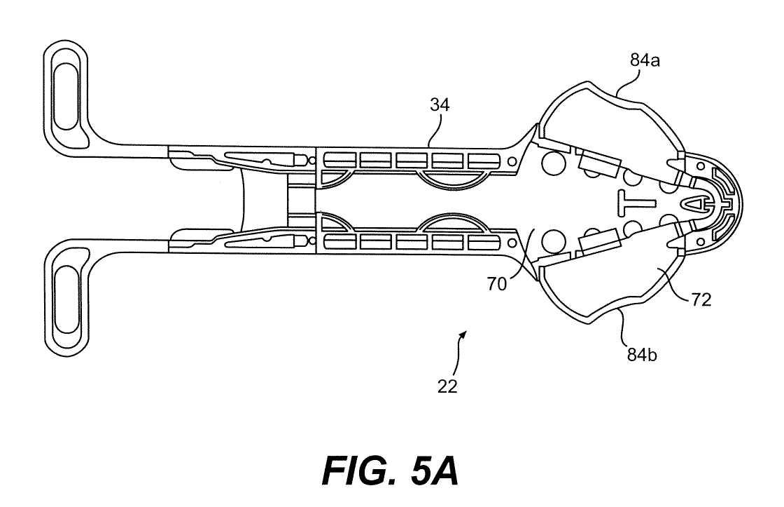

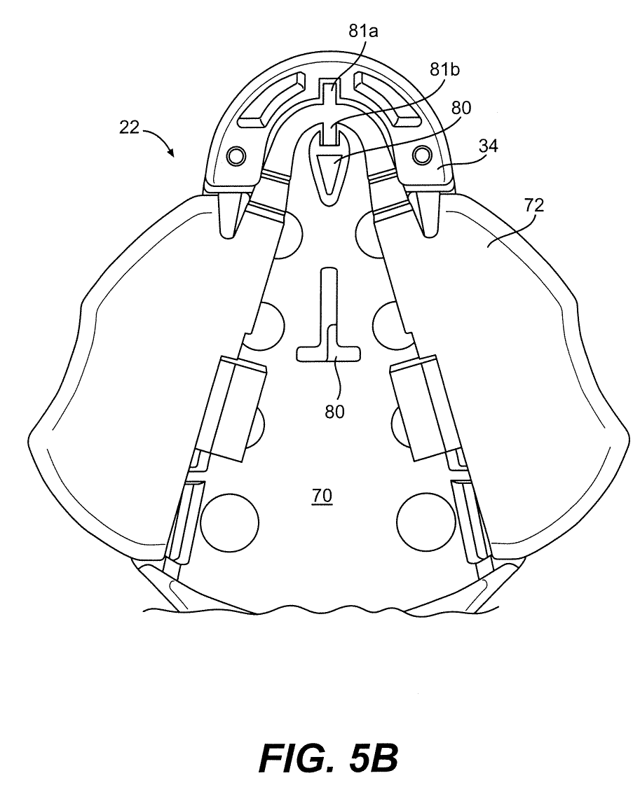

A folding mechanism can be positioned in seal housing area 70. In one aspect, the seal folding mechanism can mate with loading tool 22. For example, the folding mechanism can mechanically mate, frictionally engage, and/or adhere to the inner surface of the loading tool. Alternatively, the folding mechanism can be formed integrally with the loading tool housing. FIGS. 5A and 5B illustrate loading tool 22 with a folding mechanism mated proximate to seal housing area 70.

As mentioned above, the loading tool is configured to fold the seal into a configuration for inserting into the delivery device. Generally, the folding mechanism can include at least one articulating folding member that can change the seal from an unfolded configuration to a folded configuration. A variety of seal folding mechanisms can be used with the systems described herein, including those disclosed in co-pending applications entitled “ANASTOMOTIC SEAL LOADING TOOL,” filed even day herewith and “METHODS AND DEVICES FOR LOADING TEMPORARY HEMOSTATIC SEALS,” filed even day herewith.

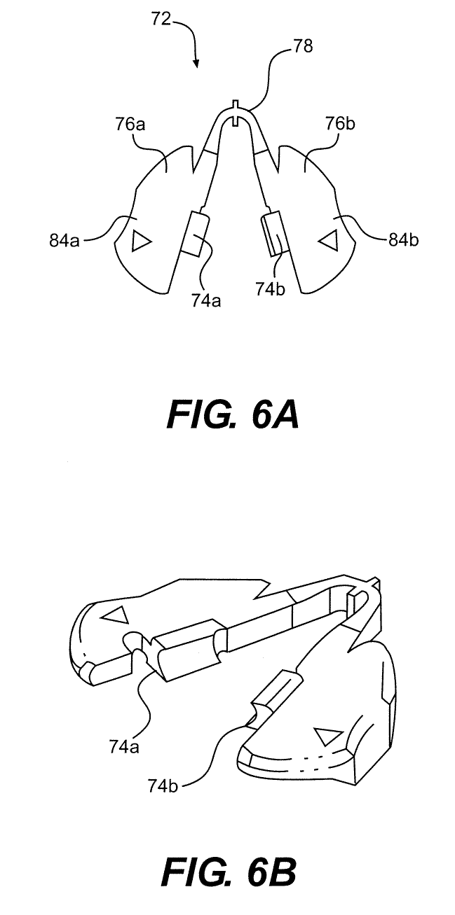

In one exemplary embodiment, loading tool 22 can include a folding mechanism defined by jaw 72. FIGS. 6A and 6B illustrate jaw 72 having a first and second seal contact area 74a, 74b. Contact areas 74a, 74b and loading tool housing 34 can define the seal housing area 70 and, where seal 26 is shipped inside loading tool 22, can hold and protect the seal during shipping and transport to the end user.

Contact areas 74a, 74b can be positioned on first and second jaw members 76a, 76b. Prior to folding, jaw members 76a, 76b can be spaced sufficiently to allow seal 26 to reside therebetween in a substantially unfolded configuration. A user can then articulate jaw 72 and cause jaw members 76a, 76b to converge. In one aspect, both jaw members can articulate relative to loading tool body 34.

Contact areas 74a, 74b, in one aspect, can have surfaces shaped to assist with folding seal 26. As the jaw members converge on seal and apply pressure to the seal, the surfaces can facilitate overlap of two, non-adjacent edges of the seal. In one exemplary embodiment, the contact surfaces have a concave or curved shape.

Jaw members 76a, 76b can be movably mated or integrally formed with one another. For example, a joint 78 can connect jaw members 76a, 76b and allow the jaw members to move relative to one another. A variety of joints can be used with the folding mechanism including. For example, where jaw members 76a, 76b are defined by a single piece structure, a living hinge can allow articulation between jaw members 76a, 76b.

As mentioned above, the folding mechanism can mate with the loading tool. Referring again to FIGS. 5A and 5B, jaw 72 can include tabs 81a, 81b that mechanically engage the inner surface of loading tool 22. For example, the tabs can mate with recesses formed in body 34 of loading tool 22. In use, tabs 81a, 81b can limit relative movement between jaw 72 and loading tool body 34, while allowing articulation of jaw members 76a, 76b. It should be appreciated that the seal folding mechanism can mate with loading tool housing in a variety of different ways depending on the chosen folding mechanism, the intended use of the system 20, and/or the configuration of loading tool 22.

Loading tool 22 can further include a folding mechanism alignment member 80 to align the folded seal with the delivery device. In one aspect, jaws are positioned around alignment member 80, such that the alignment member is positioned between jaw members 76a, 76b. Alignment member 80 can help to align the jaw members when closed by preventing, for example, the jaws from converging at a location that is offset from the axis of the delivery device lumen. Aligning the jaws with the delivery device aligns the folded seal with the delivery device.

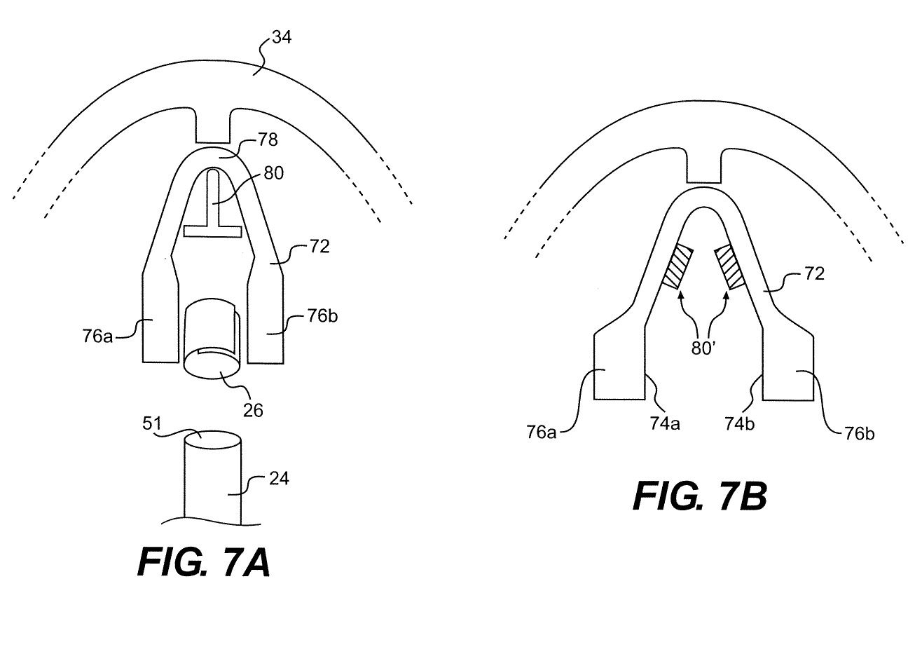

FIG. 7A illustrates folded seal 26 positioned for insertion into the lumen of delivery device 24. As shown, when jaw 72 is fully closed the “T” shaped mating member contacts the inner surface of jaw member 76a and jaw member 76b. In order to fully close jaw 72, the inner surfaces of jaw members 76a, 76b have to contact the alignment member. Thus, when a user squeezes jaws 72 to their full stop, the alignment member aligns the delivery device and the folded seal. In particular, mating member 80 centers jaw 72 when closed around mating member 80.

While alignment member 80 is illustrated as “T” shaped, alternative shapes are also contemplated. For example, the alignment member could have any shape that corresponds to the spacing between the jaws when closed. FIG. 7B illustrates, for example, a two-piece alignment member 80′ that contacts the inner surface of jaw members 76a, 76b when closed.

In one embodiment, alignment member 80 can additionally, or alternatively, act as a stop to limit movement of jaw members 76a and/or 76b. Once seal 26 is folded, further convergence of contact areas 74a, 74b can be limited by alignment member 80 to prevent damage or permanent deformation of seal 26. In one aspect, the alignment member 80 has a width that, when positioned between jaw members 76a, 76b, prevents complete convergence. However, loading tool 22 need not have a stop to control movement of the folding mechanism.

Once seal 26 is folded and aligned with the delivery device, delivery device 24 can be advanced to insert folded seal 26 into lumen 51 (FIG. 7A) of the delivery device. The folding mechanism can then be released and the delivery device withdrawn from the loading tool. In one aspect, folded seal 26 is inserted only partially into lumen 51. For example, as explained above, the loading tool can include a hard stop to limit the distal movement the delivery device with respect to the loading tool. In one aspect, the hard stop controls the distance which the folded seal is advanced into the inner lumen of the delivery device. For example, where approximately half of the folded seal is to be inserted into lumen 51, the hard stop can limit the travel of the delivery device to a depth that would achieve the desired amount of insertion.

In one aspect, at least a portion of the seal as folded by the folding mechanism of loading tool 22 can have a diameter larger than the inner diameter of lumen 51. The size of the folded seal can permit the seal to mate with lumen 51 via a frictional or interference fit.

In one aspect, the folded seal has a maximum diameter than is equal to or smaller than the outer diameter of the delivery device sheath. As the delivery device is removed from the loading tool, the size of the folded seal reduces the chance of the seal dislodging from the delivery device. While the folded seal may contact the inner surface of the loading tool, the dimensions of the folded seal can prevent the loading tool from damaging or dislodging the seal from lumen 51. In another aspect, the folded seal can have maximum diameter equal to or smaller than the smallest cross-sectional dimension of channel 62. The smaller diameter of the folded seal allows removal of the folded seal from the loading tool without obstruction by the alignment features in channel 62 of loading tool 22.

In one aspect, with respect to FIGS. 5A and 6A, jaw 72 includes user contact surfaces 84a, 84b for control of the folding mechanism. Jaw 72 can have a width that allows the jaw to extend through the sidewall of housing 34, such that the user contact surfaces 84a, 84b are accessible by a user, while the inner, seal contact areas 74a, 74b are positioned inside housing 34. A user can apply pressure on the jaw members, via user contact surfaces 84a, 84b, to fold seal 26. Once the seal is at least partially positioned within delivery device 24, a user can release jaw 72. In one aspect, the jaw 72 is biased in the open position and release of contact surfaces 84a, 84b allows seal contact surfaces 74a, 74b to move away from one another.

In another aspect, jaw 72 can be fully contained within housing 34. For example, a user can fold the seal by pressing on a flexible wall which directs force to jaw 72. Alternatively, a lever, button, or other user control mechanism can be positioned between jaw 72 and the outer surface of housing 34. It should also be appreciated that where the folding mechanism comprises a single articulating folding member, a single user contact surface can be provided.

While the Figures illustrate a pivoting or jaw-like folding mechanism, other configurations can be used with the loading tool described herein. In one aspect, the folding mechanism can have parallel, opposed seal contact surfaces, at least one of which moves or slides toward the other. In another aspect, instead of pivoting around an axis that is generally orthogonal to a longitudinal axis of the loading tool, one or both of the seal contact areas 74a, 74b can pivot relative to an axis that is generally parallel to the central longitudinal axis of the loading mechanism.

In addition, while the seal contact areas 74a, 74b are described as folding the seal, other portions of the loading tool can assist with folding. For example, the inner surface of the loading tool housing adjacent to the seal housing area can work with seal contact area 74a, 74b to fold the seal.

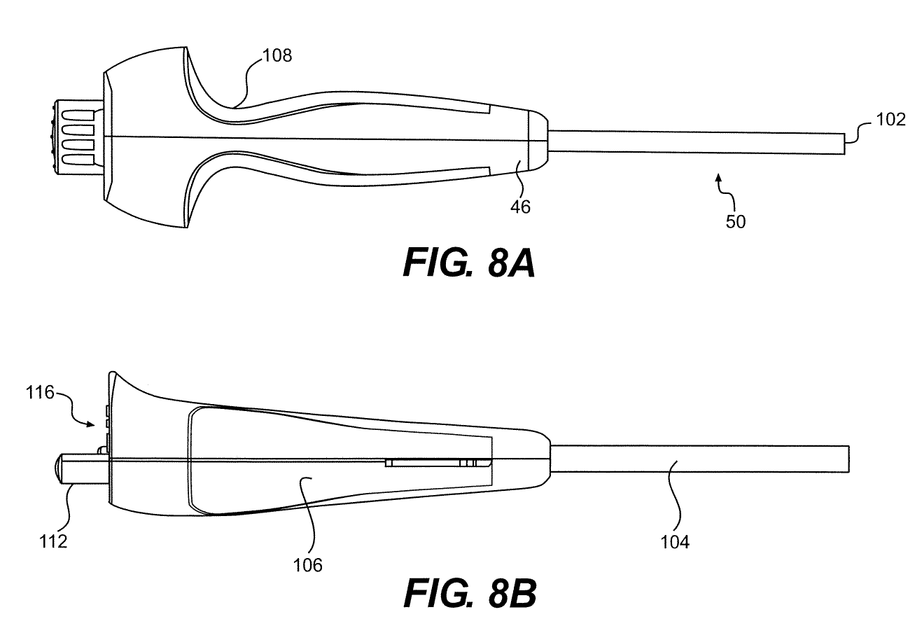

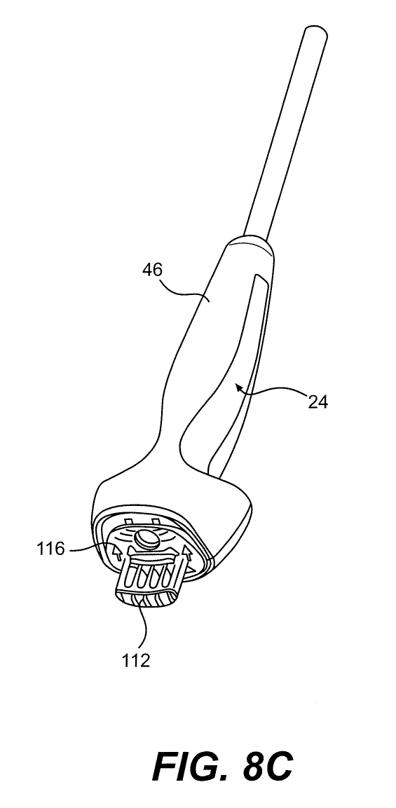

Further described herein are various embodiments of the delivery device. The delivery device can have a variety of shapes and sizes, however as described above, the outer surface of delivery device 24 can, at least in part, be configured for receipt in the loading tool. FIGS. 8A through 8C illustrate several views of delivery device 24. A distal portion of the delivery device body 46 can have an opening 102 for receipt of folded seal 26. In addition, opening 102 can provide ingress into a inner lumen 51 (FIG. 7A) defined by a distal body member 104. While opening 102 is illustrated at the distal-most end of the delivery device the opening could be located at a more proximal location.

In one aspect, distal body member 104 has a generally cylindrical configuration. For example, the distal body member can be defined by an elongate tube or sheath having a size and shape configured for passage through channel 62 of loading tool 22. In particular, the distal body member can be shaped for passage through alignment members 66 of the loading tool. Depending on the configuration of loading tool 22 and/or seal 26, the distal body member can have a variety of alternative shapes.

A proximal portion of delivery device body 46 can have a generally larger cross-sectional area than distal body member 104. The larger width of a proximal body portion 106 can mate with a hard and/or soft stop of the loading tool 22 as described above. Alternatively, or additionally, a distal portion of body 46 could include a shape configured to mate with a hard or soft stop of the loading tool.

Proximal body member 106 can act as a handle for grasping by a user. In one exemplary embodiment, the proximal body member 106 includes a flared distal end to allow a user to grasp a recessed area 108. In addition, or alternatively, a user can contact the distal-most end of the delivery device body 46 to move the delivery device with respect to the loading tool. One skilled in the art will appreciate that a variety of differently shaped distal body portions can allow a user to grasp and/or control movement of the delivery device with respect to the loading tool.

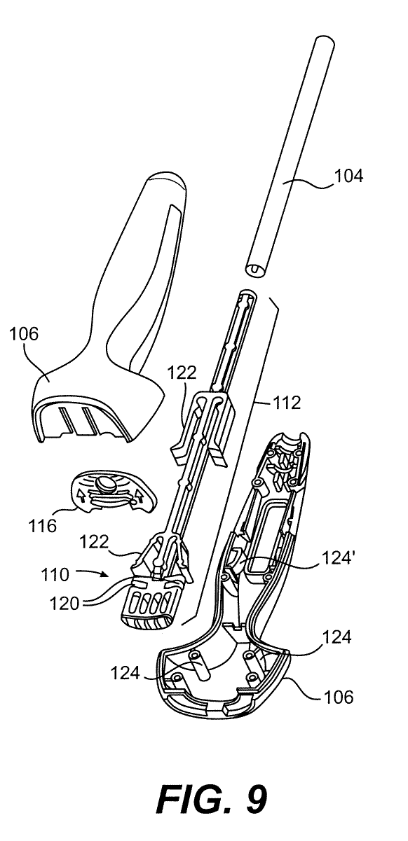

FIG. 9 illustrates an exploded view of delivery device 24. Proximal body member 106 can comprise a single or multi-piece structure for housing and/or mating with distal body member 104. In addition, the delivery device can include a plunger 112 to discharge a folded seal from the distal end of delivery device 24. In one aspect, plunger 112 includes a proximal end and a distal end for contacting the seal 26. At least a portion of plunger 112 is sized and shaped for receipt in the distal body member 104 and can move with respect to the distal body member. The proximal end of the plunger can extend proximally of the proximal body member 106 and allow a user to control seal delivery. To discharge the folded seal, a user can apply force on the proximal end of the plunger and move the plunger distally.

In one aspect, delivery device 24 includes a locking mechanism to prevent accidental discharge of the folded seal from distal body member 104. In one aspect, the distal end of delivery device 24 can include a locking mechanism 110, that when engaged, inhibits movement of the plunger and/or unwanted discharge of the folded seal. In addition, or alternatively, delivery device 24 can include tactile feedback that indicates to a user when the plunger has been fully engaged.

The locking mechanism, in one exemplary aspect, can be defined, at least in part, by opposed grooves 120 that are configured to mate with a portion of the delivery device housing. In one exemplary embodiment, locking mechanism 110 can move between a first and a second position. In the first position, the locking mechanism allows movement of the plunger. For example, when “unlocked” a user can move the plunger longitudinally within the lumen of the delivery device. Conversely, in the second position, the locking mechanism can engage the body 46 of the delivery device and inhibit movement of the plunger. The locking mechanism can mate with a variety of different parts of the delivery device, but in one aspect, mates with a proximal wall 116 and/or proximal body member 106.

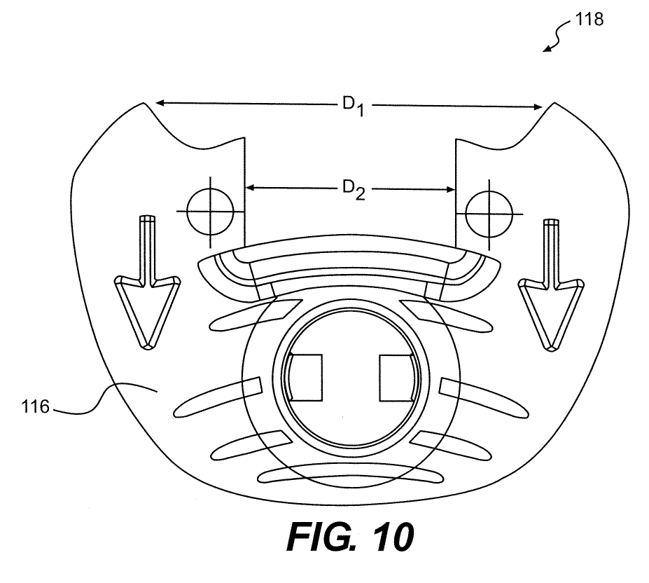

FIG. 10 illustrates a front view of distal wall 116 having a passageway 118 through which plunger 112 and locking mechanism 110 can pass. Passageway 118 includes a first dimension D₁ and a second, smaller dimension D₂. Plunger can move through dimension D₁, while the smaller size of the dimension D₂ allow the locking mechanism to engage distal wall 116. Thus, in the first position, the locking member can be positioned in an opening having a width greater then the width of the plunger. Conversely, in the second position, grooves 120 can mate with distal wall 116 and prevent distal movement of the plunger. In use, a clinician can move the distal end of the plunder up/down to unlock/lock the plunger with respect to the body of the delivery device.

In another embodiment, instead of grooves, or in addition thereto, plunger 112 could have a width or protrusion that engages the body of the delivery device 24. In a manner similar to that described above, a passageway 118 can limit or allow movement of the protrusion therethrough. For example, the passageway can include a first width larger than the protrusion and a second width smaller than the protrusion. To move the plunger 112 through delivery device 24, the protrusion can be aligned with the larger width of the protrusion. In still another embodiment, the size (e.g., width) of the passageway 118 and/or locking mechanism can be controlled. For example, instead of moving the plunger relative to wall 116, the user could change the size of passageway 118.

While the movement of plunger 112 and locking mechanism 110 is described as up/down, in another aspect, movement of the plunger between the locked and unlock configuration can be achieved with a side-to-side or rotational movement.

Delivery device 24, as mentioned above, can include feedback to alert a user to the location of the plunger within the delivery device and/or that the plunger has deployed the seal. As the plunger moves through the delivery device body 46, plunger 112 can engage the inner surface of the delivery device. In one aspect, the plunger can engage the delivery device body in a mechanical and/or fit. For example, as shown in FIG. 9, the plunger can include at least one flexible and/or resilient member 122 that can engage a pin 124 as the plunger moves within the delivery device. Flexible member can snap past the pin and create tactile feedback when the plunger reaches a chosen location. For example, the flexible member can snap past the pin after or at the same time as the distal end of the plunger reaches a point that will cause the seal to deploy from the delivery device. In one aspect, multiple flexible members 122 can engage multiple pins 124.

In another aspect, a protrusion or recess on proximal body 106 can be used instead of, or in addition to, pin 124. For example, as shown in FIG. 9, protrusion 124′ can provide tactile feedback when member 122′ moves past the protrusion. Where more than one feedback mechanism is provided, user feedback can be provided at different times. For example, as a user begins to move the plunger a first tactile indication can be delivered. When the plunger reaches a location that will dislodge a seal, a second tactile indication can be provided.

The delivery device can also include a soft stop similar to the soft stops described with respect to the loading tool. For example, the force required to move the flexible member 122 past pin 124 can create a soft stop.

In yet another embodiment, the delivery device is a single use device. The plunger can be configured to allow distal movement, but once plunger 112 has been moved to its deployment configuration, the delivery device is configured to inhibit return to the seal insertion configuration. The flexible members 122, 122′, for example, once moved passed pins 124 or protrusions 124′ can prevent proximal movement of the plunger within the delivery device.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device delivery system, comprising:
an anatomic seal comprising a flexible body;
a loading tool comprising a body having a proximal opening sized for receiving at least a portion of a delivery device and a seal folding area sized and shaped for housing a substantially unfolded seal, the loading tool further comprising a folding mechanism comprising at least one articulating member having a seal contact surface; and
a delivery device having an elongate body and an open interior for receiving a folded seal and a plunger for deploying the seal, the delivery device and the loading tool adapted to detachably mate with one another.

2. The system of claim 1, wherein the outer surface of the delivery device includes a first mating feature and the inner surface of the loading tool includes a corresponding second mating feature.

3. The system of claim 1, wherein the loading tool includes at least one alignment feature to prevent relative transverse movement between the loading tool and the delivery device while allowing relative longitudinal movement.

4. The system of claim 3, wherein the at least one alignment feature aligns the elongate body of the delivery device with the seal when the seal is an a folded configuration.

5. The system of claim 1, wherein the loading tool includes at least one hard stop to limit advancement of the delivery device into the loading tool.

6. The system of claim 1, wherein the opening in the loading tool defines an opening into a channel configured for receipt of at least a portion of the elongate body of the delivery device.

7. The system of claim 6, wherein the channel allows passage of at least a portion of the elongate body and does not allow passage of the seal in an unfolded configuration.

8. The system of claim 7, wherein the channel allows passage of the seal in a folded configuration.

9. The system of claim 1, further comprising a soft stop to inhibit relative movement of the delivery device with respect to the loading tool.

10. A hemostatic seal delivery system, comprising:
a delivery device having an elongate body extending between a proximal handle and a distal opening for receiving the hemostatic seal in a folded configuration, the delivery device further comprising an inner surface and an outer surface; and
a loading tool comprising a body having a proximal opening and a channel for receiving at least a portion of the delivery device body, the channel having at least one dimension that corresponds to a dimension of the outer surface of the delivery device body, the loading tool including a seal housing area comprising a folding mechanism including at least one articulating member, wherein the loading tool channel extends between the proximal opening and the seal housing area.

11. The system of claim 10, wherein the loading tool channel has a shape corresponding to at least a portion of the delivery device body.

12. The system of claim 10, wherein the loading tool channel includes a soft stop adapted to inhibit relative movement between the loading tool and the delivery device.

13. The system of claim 12, wherein the soft stop is selected from the group consisting of a snap fit, an interference fit, frictional engagement, and combinations thereof.

14. The system of claim 12, wherein the soft stop inhibits distal movement of the delivery device with respect to the loading tool.

15. The system of claim 12, wherein the soft stop inhibits withdrawal of the delivery device with respect to the loading tool.

16. The system of claim 10, further comprising at least two soft stops.

17. The system of claim 10, further comprising a hard stop the prevents distal movement of the delivery device with respect to the loading tool.

18. The system of claim 10, further comprising a hemostatic seal.

19. The system of claim 18, wherein the channel is sized for passage of delivery device, but not the hemostatic seal in its unfolded configuration.

20. A hemostatic seal delivery device, comprising:
an elongate body extending between a proximal handle and a distal opening, the elongate body including an inner lumen for receiving the hemostatic seal in a folded configuration, the delivery device further comprising a plunger extending through at least a portion of the lumen and movably mated with the elongate body; and
a locking mechanism defined by at least a portion of the elongate body or plunger and configured to prevent movement of the plunger with respect to the lumen when locked and configured to allow movement of the plunger with respect the lumen when unlocked.

21. The device of claim 20, wherein the locking mechanism comprises a groove on the plunger adapted to mate with the elongate body.

22. A loading device comprising:
a body having a proximal opening and a channel for receiving at least a portion of a delivery device body, the loading tool including a seal housing area for containing a hemostatic seal in a substantially unfolded configuration, wherein the loading tool channel extends between the proximal opening and the seal housing area, the loading tool further comprising a folding mechanism including at least one articulating member, wherein the channel is shaped and sized to prevent passage of an unfolded hemostatic seal.

23. The device of claim 22, wherein the articulating member comprises a jaw having first and second jaw members.

24. The device of claim 23, wherein an unfolded hemostatic seal is positioned between the first and second jaw members.

25. A method for delivering a hemostatic seal, comprising:
providing a delivery device having an elongate body extending between a proximal handle and a distal opening for receiving the hemostatic seal in a folded configuration;
providing a loading tool comprising a channel for receiving and detachably mating with at least a portion of the delivery device body, the loading tool including a hemostatic seal housing area containing the hemostatic seal in a substantially unfolded configuration and a folding mechanism including at least one articulating member;
folding the hemostatic seal by moving the at least one articulating member relative to the seal housing area; and
advancing the delivery device into seal housing area to insert the folded hemostatic seal at least partially into the distal opening.

26. The method of claim 25, further comprising the step of withdrawing the delivery device from the loading tool.

27. The method of claim 25, wherein the step of folding includes folding the seal into an oblate spheroid shape.

28. The method of claim 25, wherein the step of advancing includes advancing the delivery device until the delivery device encounters a hard stop in the loading tool.

29. The method of claim 25, wherein the articulating member includes a jaw having two jaw members.

30. The method of claim 29, wherein the folding stop includes converging the jaw members.

31. The method of claim 29, further comprising the step of moving the jaw members apart from one another prior to withdrawing the delivery device from the loading tool.
